(19) United States
(12) Patent Application Publication
(10) Pub. No.: US 2008/0234562 A1
(43) Pub. Date: Sep. 25, 2008

(54) Continuous analyte monitor with multi-point self-calibration

(76) Inventor: Arvind N. Jina, San Jose, CA (US)

Correspondence Address: SHAY GLENN LLP, 2755 CAMPUS DRIVE, SUITE 210, SAN MATEO, CA, 94403, US

(21) Appl. No.: 11/725,624

(22) Filed: Mar. 19, 2007

(51) Int. Cl. A61B 5/1495

(52) U.S. Cl. 600/365

(57) ABSTRACT

Analyte monitors and their methods of use. The analyte monitors include multiple calibration fluids which may have different known concentrations of an analyte, such as glucose. The analyte monitors may also include sensing or washing fluids. The analyte monitors are configured to be calibrated with the multiple calibration fluids to potentially provide a more accurate determination of analyte concentrations. The analyte monitors can be adapted to be self-calibrating with the multiple calibration fluids.

### BACKGROUND OF THE INVENTION

The invention relates to systems, devices, and tools, and the use of such systems, devices and tools for monitoring an analyte or analytes, such as glucose levels in a person having diabetes. More specifically, the invention relates to systems, devices, and tools and the use of such systems, devices and tools for monitoring analyte levels continuously, or substantially continuously.

Diabetes is a chronic, life-threatening disease for which there is no known cure at present. It is a syndrome characterized by hyperglycemia and relative insulin deficiency. Diabetes affects more than 120 million people world wide, and is projected to affect more than 220 million people by the year 2020. It is estimated that one out of every three children today will develop diabetes sometime during their lifetime. Diabetes is usually irreversible, and can lead to a variety of severe health complications, including coronary artery disease, peripheral vascular disease, blindness and stroke. The Center for Disease Control (CDC) has reported that there is a strong association between being overweight, obesity, diabetes, high blood pressure, high cholesterol, asthma and arthritis. Individuals with a body mass index of 40 or higher are more than 7 times more likely to be diagnosed with diabetes.

There are two main types of diabetes, Type I diabetes (insulin-dependent diabetes mellitus) and Type II diabetes (non-insulin-dependent diabetes mellitus). Varying degrees of insulin secretory failure may be present in both forms of diabetes. In some instances, diabetes is also characterized by insulin resistance. Insulin is the key hormone used in the storage and release of energy from food.

As food is digested, carbohydrates are converted to glucose and glucose is absorbed into the blood stream primarily in the intestines. Excess glucose in the blood, e.g. following a meal, stimulates insulin secretion, which promotes entry of glucose into the cells, which controls the rate of metabolism of most carbohydrates.

Insulin secretion functions to control the level of blood glucose both during fasting and after a meal, to keep the glucose levels at an optimum level. In a non-diabetic person blood glucose levels are typically between 80 and 90 mg/dL of blood during fasting and between 120 to 140 mg/dL during the first hour or so following a meal. For a person with diabetes, the insulin response does not function properly (either due to inadequate levels of insulin production or insulin resistance), resulting in blood glucose levels below 80 mg/dL during fasting and well above 140 mg/dL after a meal.

Currently, persons suffering from diabetes have limited options for treatment, including taking insulin orally or by injection. In some instances, controlling weight and diet can impact the amount of insulin required, particularly for non-insulin dependent diabetics. Monitoring blood glucose levels is an important process that is used to help diabetics maintain blood glucose levels as near as normal as possible throughout the day.

The blood glucose self-monitoring market is the largest self-test market for medical diagnostic products in the world, with a size of approximately over $3 billion in the United States and $7.0 billion worldwide. It is estimated that the worldwide blood glucose self-monitoring market will amount to $9.0 billion by 2008. Failure to manage the disease properly has dire consequences for diabetics. The direct and indirect costs of diabetes exceed $130 billion annually in the United States—about 20% of all healthcare costs.

There are two main types of blood glucose monitoring systems used by patients: non-continuous, also known as single point, discrete or episodic, and continuous. Non-continuous systems consist of meters and tests strips and require blood samples to be drawn from fingertips or alternate sites, such as forearms and legs (e.g. OneTouch® Ultra by LifeScan, Inc., Milpitas, Calif., a Johnson & Johnson company). These systems rely on lancing and manipulation of the fingers or alternate blood draw sites, which can be extremely painful and inconvenient, particularly for children.

Continuous monitoring sensors are generally implanted subcutaneously and measure glucose levels in the interstitial fluid at various periods throughout the day, providing data that shows trends in glucose measurements over a short period of time. These sensors are painful during insertion and usually require the assistance of a health care professional. Further, these sensors are intended for use during only a short duration (e.g., monitoring for a matter of days to determine a blood sugar pattern). Subcutaneously implanted sensors also frequently lead to infection and immune response complications. Another major drawback of currently available continuous monitoring devices is that they require frequent, often daily, calibration using blood glucose results that must be obtained from painful finger-sticks using traditional meters and test strips. This calibration, and re-calibration, is required to maintain sensor accuracy and sensitivity, but it can be cumbersome and inconvenient.

At this time, there are four products approved by the FDA for continuous glucose monitoring, none of which are presently approved as substitutes for current glucose self-monitoring devices. Medtronic (www.medtronic.com) has two continuous glucose monitoring products approved for sale: Guardian® RT Real-Time Glucose Monitoring System and CGMS® System. Each product includes an implantable sensor that measures and stores glucose values for a period of up to three days. One product is a physician product. The sensor is required to be implanted by a physician, and the results of the data aggregated by the system can only be accessed by the physician, who must extract the sensor and download the results to a personal computer for viewing using customized software. The other product is a consumer product.

A third product approved for continuous glucose monitoring is the Glucowatch® developed by Cygnus Inc., which is worn on the wrist like a watch and can take glucose readings every ten to twenty minutes for up to twelve hours at a time. It requires a warm up time of 2 to 3 hours and replacement of the sensor pads every 12 hours. Temperature and perspiration are also known to affect its accuracy. The fourth approved product is a subcutaneously implantable glucose sensor developed by Dexcom, San Diego, Calif. (www.dexcom.com). All of the approved devices are known to require daily, often frequent, calibrations with blood glucose values which the patient must obtain using conventional finger stick blood glucose monitors.

### SUMMARY OF THE INVENTION

The invention involves an analyte monitor that may be periodically calibrated with a minimum number or no finger sticks or other painful invasive calibration techniques and measures an analyte such as glucose without drawing any interstitial fluid (or any other fluid) from the user.

One aspect of the invention is an analyte monitor. The analyte monitor includes a plurality of tissue piercing elements each having a distal opening, a proximal opening, and an interior lumen extending between the distal and proximal openings, a sensing area in fluid communication with the proximal openings of the plurality of tissue piercing elements, a plurality of calibration fluid reservoirs each adapted to house a calibration fluid, wherein the plurality of calibration fluid reservoirs are in fluid communication with the sensing area, and a sensor configured to detect an analyte and provide an output indicative of the concentration of the analyte in a fluid in the sensing area.

In some embodiments the plurality of calibration fluid reservoirs include a first calibration fluid reservoir adapted to house a first calibration fluid and a second calibration fluid reservoir adapted to house a second calibration fluid. The first and second calibration fluids can have different known concentrations of an analyte, such as between about 0 mg/dl and about 100 mg/dl and between about 100 mg/dl and about 400 mg/dl of glucose respectively.

In some embodiments the monitor further includes an actuator, such as a pump configured to move the calibration fluids from the plurality of calibration fluid reservoirs into the sensing area. The monitor can include a plurality of valves configured to facilitate the movement of the calibration fluids from the plurality of calibration fluid reservoirs into the sensing area. The actuator can be configured to be manually or automatically actuated.

In some embodiments the monitor also includes a programmable component in communication with the actuator where the programmable component is programmed to automatically actuate the actuator.

The monitor may also include a remote device. A programmable component can be disposed in a housing with the sensor or it can be disposed in the remote device. The programmable component can be configured to be wirelessly programmed using the remote device. The programmable component can also be configured to be in wireless communication with the actuator to automatically actuate the actuator.

In some embodiments the actuator is configured to move a first calibration fluid with a first known analyte concentration from a first calibration fluid reservoir into the sensing area and then move a second calibration fluid with a second known analyte concentration from a second calibration fluid reservoir into the sensing area, thereby displacing the first calibration fluid from the sensing area. The sensor can be configured to detect the analyte in the first and second calibration fluids when in the sensing area, where the monitor also includes a memory to store a sensor calibration, which can be disposed in a remote device. In some embodiments the sensor calibration includes the first and second known analyte concentrations and a first output and a second output from the sensor indicative of the first and second known analyte concentrations.

The monitor may also include a transmitter configured to transmit an output from the sensor indicative of the amount of analyte, such as glucose, that has diffused from the patient's interstitial fluid into the sensing area to a receiver disposed in a remote device, the remote device further comprising a processor adapted to determine an analyte concentration based on the output from the sensor and the sensor calibration values stored in the memory. The transmitter can be either fabricated without a power source or it comprises a rechargeable power source.

The monitor can include a display, which can be disposed in the remote device, adapted to display the analyte concentration determined by the processor. The displayed analyte concentration can be the patient's blood glucose level.

The monitor may also include at least one waste reservoir in fluid communication with the sensing area adapted to receive fluid moved from the sensing area.

The monitor may include a housing including a disposable portion and reusable portion, the disposable portion being adapted to support the plurality of tissue piercing elements, the plurality of calibration fluid reservoirs, the sensing area, and at least part of the analyte sensor, the reusable portion including an electrical connection to the at least part of the analyte sensor in the disposable portion, the housing further comprising a connector adapted to connect and disconnect the disposable portion from the reusable portion.

The monitor may include a sensing fluid reservoir in fluid communication with the sensing area, where the sensing fluid reservoir is adapted to house a sensing fluid which does not comprise an analyte, such as buffer, surfactants or preservatives.

In some embodiments the monitor also includes a transmitter adapted to transmit the output indicative of the analyte concentration of the fluid in the sensing area to a remote device, at least one power source, a reusable portion comprising the transmitter, a disposable portion comprising the at least one power source, where the at least one power source is adapted to be disposable and wherein the transmitter is adapted to be reusable.

One aspect of the invention is a method of monitoring a patient's interstitial fluid analyte concentration in vivo. The method includes calibrating an analyte monitor. Calibrating the analyte monitor includes moving a first calibration fluid with a first known analyte concentration from a first calibration reservoir into the sensing area, sensing an analyte concentration in the first calibration fluid while in the sensing area in contact with the analyte sensor, the sensor providing a first output indicative of the analyte concentration of the first calibrating fluid, moving a second calibration fluid with a second known analyte concentration from a second calibration reservoir into the sensing area thereby displacing the first calibration fluid with the second calibration fluid into the at least one waste reservoir and sensing an analyte concentration in the second calibration fluid while in the sensing area in contact with the analyte sensor, the sensor providing a second output indicative of the analyte concentration of the second calibrating fluid, and storing sensor calibration values or readings in the memory, the sensor calibration comprising an association between the first and second known analyte concentrations and the first and second outputs indicative of the first and second known analyte concentrations.

In some embodiments the second calibration fluid is a sensing fluid that does not include an analyte, and moving the sensing fluid into the sensing area includes washing the sensing area with the sensing fluid.

In some embodiments the method also comprises piercing only as deep as into the epidermis layer of a patient's skin with the plurality of tissue piercing elements. In these embodiments, piercing only as deep as into the epidermis layer of the patient's skin with the plurality of tissue piercing elements allows diffusion of an analyte from the patient's interstitial fluid through the plurality of tissue piercing elements and into the sensing area substantially without extracting interstitial fluid through the plurality of tissue piercing elements.

In some embodiments the method further comprises sensing the analyte concentration of the diffused analyte using the sensor and determining the patient's analyte concentration using the sensor calibration stored in the memory.

In some embodiments the monitor also includes a remote device, and where the memory is disposed in the remote device, the method further includes wirelessly transmitting the outputs from the sensor to the remote device before determining the patient's analyte concentration. The method may include displaying the determined analyte concentration, such as using the remote device.

In some embodiments the method includes moving a sensing fluid which does not include an analyte from a third sensing fluid reservoir into the sensing area thereby displacing the second calibration fluid with the sensing fluid into the at least one waste reservoir, where moving the sensing fluid occurs before the piercing step.

In some embodiments the method further includes recalibrating the sensor after determining the presence or concentration of an analyte or analytes in the patient.

In some embodiments moving the first and second calibration fluids comprises actuating an actuator, which can be done automatically or manually. The monitor may be programmed to automatically actuate the actuator. The monitor may include a remote device, and a software program to automatically actuate the actuator using the remote device.

Other embodiments of the invention will be apparent from the specification and drawings.

### Incorporation by Reference

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view of one embodiment of the analyte monitor wherein the monitor comprises a plurality of calibration fluid reservoirs.

FIG. 2 is a cross-sectional view showing exemplary components of an analyte monitor on a patient with tissue piercing elements piercing through the patient's skin

FIGS. 3 and 4 illustrate one embodiment in which the analyte monitor comprises a plurality of calibration fluid reservoirs and a sensing fluid reservoir.

FIG. 5 shows an exploded view of an analyte monitor according to one embodiment of the invention.

FIGS. 6A and 6B are a schematic representative drawing of a three electrode system for use with the analyte sensor of one embodiment of this invention. FIG. 6A shows electrodes on a substrate, and FIG. 6B shows the electrodes and a portion of the substrate covered with a reagent.

FIGS. 7A and 7B are a schematic representative drawing of a two electrode system for use with the analyte sensor of one embodiment of this invention. FIG. 7A shows electrodes on a substrate, and FIG. 7B shows the electrodes and a portion of the substrate covered with a reagent.

FIG. 8 is a cross-sectional schematic view of a portion of an analyte monitoring device wherein an actuator is disposed on the side of the device.

FIG. 9 shows a remote device with a display and user controls for use with an analyte monitoring system according to yet another embodiment of the invention.

FIG. 10 shows an analyte sensor in place on a patient's skin and a remote device for use with the sensor.

### DETAILED DESCRIPTION OF THE INVENTION

While many of the exemplary embodiments disclosed herein are described in relation to monitoring glucose levels in people with diabetes, it should be understood that aspects of the invention are useful in monitoring glucose levels in people without diabetes, or for monitoring an analyte or analytes other than glucose. For example, the present invention may be used in monitoring the concentration, or presence, of other analytes such as lactate, acetyl choline, amylase, bilirubin, cholesterol, chorionic gonadotropin, creatine kinase (e.g., CK-MB), creatine, DNA, fructosamine, glutamine, growth hormones, hematocrit, hemoglobin (e.g. HbAlc), hormones, ketones, lactate, oxygen, peroxide, prostate-specific antigen, prothrombin, RNA, thyroid stimulating hormone, troponin, drugs such as antibiotics (e.g., gentamicin, vancomycin), digitoxin, digoxin, drugs of abuse, theophylline, and warfarin. Accordingly, use of the word “glucose” herein may be taken to mean any analyte, depending on the context.

The present invention provides a significant advance in biosensor and analyte monitoring technology. According to various aspects of the invention, a glucose monitoring system may be constructed to be portable, painless, virtually non-invasive, self-calibrating, integrated and/or have non-implanted sensors which continuously indicate the user's glucose concentration, enabling swift corrective action to be taken by the patient. The invention may also be used in critical care situations, such an in an intensive care unit to assist health care personnel. The sensor and monitor of this invention may be used to measure any other analyte as well, for example, electrolytes such as sodium or potassium ions. As will be appreciated by persons of skill in the art, the glucose sensor can be any suitable sensor including, for example, an electrochemical sensor or an optical sensor.

One aspect of the invention is a glucose monitor. The glucose monitor may comprise a plurality of tissue piercing elements, a sensing area in fluid communication with the plurality of tissue piercing elements, a plurality of calibration reservoirs each adapted to house a calibration fluid and in fluid communication with the sensing area, and a sensor configured to detect glucose and provide an output indicative of the glucose concentration of the fluid in the sensing area.

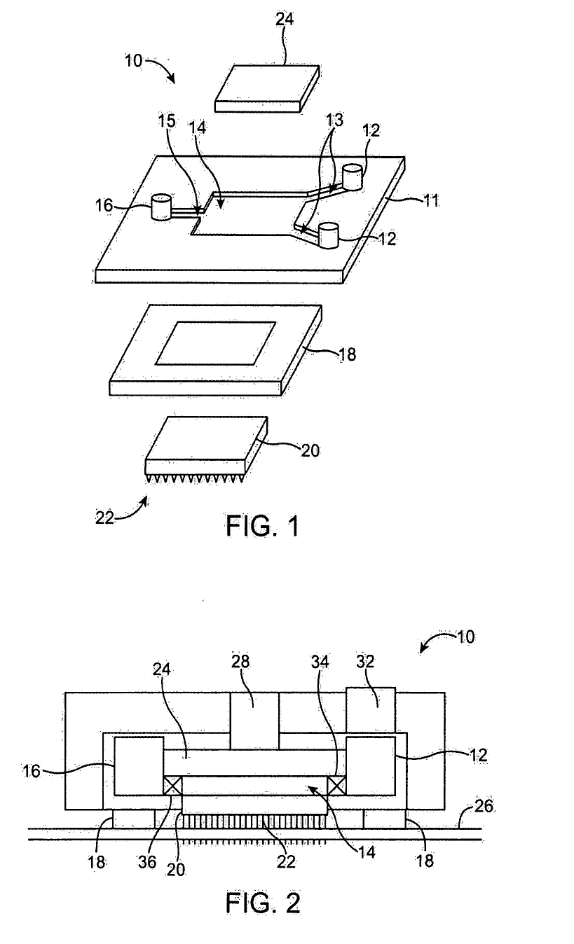

FIG. 1 illustrates one embodiment of the present invention. Glucose monitor 10 includes a fluidic network in which two calibration reservoirs 12 in fluid communication with sensing area 14 and waste reservoir 16 to allow for the movement of calibration fluids from the reservoirs through sensing area 14 and into the waste reservoir 16. Glucose monitor 10 includes adhesive pad or seal 18 which is coupled to substrate or chip 20 which comprises a plurality of tissue piercing elements 22.

As shown, glucose monitor 10 includes calibration reservoirs 12 in fluid communication with calibration fluid channels 13, which are adapted to receive calibration fluid from the calibration fluid reservoirs. Calibration fluid channels 13 are in fluid communication with sensing area or sensing channel 14. Sensing area 14 is fluidly connected via a check valve to waste channel 15, which is in fluid communication with waste reservoir 16. When substrate 20 is coupled to adhesive pad 18 and adhesive pad 18 is coupled to sensing layer 11, the plurality of piercing elements 22 are in fluid communication with sensing area 14 and with sensor 24. While not shown in FIG. 1, at least one pump and at least one check valve can be incorporated into the glucose monitor to facilitate or control the flow of fluid unidirectionally from the calibration fluid reservoirs into the sensing area. Also not shown in FIG. 1 is an actuator which can be manually or automatically actuated and can be configured to work in conjunction with a pump and/or series of valves to initiate the flow of fluid from the calibration fluid reservoirs. The channels shown in FIG. 1 are intended to be optional in the glucose monitor, as the calibration fluid can flow directly from the calibration fluid reservoirs into the sensing area (passing through valves), and further directly into the waste reservoirs. One or more waste reservoirs may be incorporated into the glucose monitor.

FIG. 2 is a side sectional view of one embodiment of the invention. The embodiment in FIG. 2 is similar to that of FIG. 1, however the channels from FIG. 1 are not present in FIG. 2. While only one calibration reservoir is shown in FIG. 2, a plurality of calibration reservoirs are present in the embodiment. The glucose monitor 10 includes tissue piercing elements 22 extending through the stratum corneum 26 of a user into the interstitial fluid beneath the stratum corneum. The tissue piercing elements are hollow and generally have open distal ends, and their interiors communicate with a sensing area 14. Sensing area 14 is therefore in fluid communication with interstitial fluid through tissue piercing elements 22. In this embodiment, sensing area 14 and the tissue piercing elements 22 are pre-filled with sensing fluid prior to the first use of the device. Thus, when the device is applied to the user's skin and the tissue piercing elements pierce the stratum corneum and the epidermis, there is substantially no net fluid transfer from the interstitial fluid into the tissue piercing elements. Rather, glucose diffuses from the interstitial fluid into the fluid within the tissue piercing elements, as described below.

Exemplary tissue piercing elements that can be used with the present invention include microneedles described in Stoeber et al. U.S. Pat. No. 6,406,638; US Patent Appl. Publ. No. 2005/0171480; and US Patent Appl. Publ. No. 2006/0025717. Tissue piercing elements and microneedles described in co-assigned U.S. patent application Ser. No. 11/642,196, filed Dec. 20, 2006 may also be used. Any other tissue piercing elements or needle arrays that can penetrate into the epidermis layer and allow glucose to diffuse from the interstitial fluid into the sensing area of the present invention may also be incorporated into the embodiments described herein.

Disposed above and in fluid communication with sensing area 14 is sensor 24. In some embodiments, the sensor is an electrochemical glucose sensor that generates an electrical signal (current, voltage or charge) whose value depends on the concentration of glucose in the fluid within sensing area 14. Details of sensor 24 are discussed in more detail below.

Electronics element 28 is configured to receive an electrical signal from sensor 24. In some embodiments, electronics element 28 uses the electrical signal to compute a glucose concentration and display it. In other embodiments, electronics element 28 transmits the electrical signal, or information derived from the electrical signal, to a remote device, such as through wireless communication. Electronics element 28 can comprise other electrical components such as an amplifier and an A/D converter which can amplify the electrical signal from the sensor and convert the amplified electrical signal to a digital signal before, for example, determining a glucose concentration or transmitting the signal to an external device which can then determine a glucose concentration.

Glucose monitor 10 can be held in place on the patient's skin by one or more adhesive pads 18.

The glucose monitor has a built-in calibration system. As shown in FIG. 1, the glucose monitor includes a plurality of calibration reservoirs each adapted to house a calibration fluid. The plurality of calibration reservoirs are in fluid communication with the sensing area. A glucose monitor with two or more calibration fluids can have a sensor that can be calibrated at two or more different glucose concentrations, which allows for a multi-point calibration curve during the sensor calibration. This can provide a more accurate calibration curve which in turn can enable a more accurate glucose concentration determination.

The calibration fluids in each of the different calibration fluid reservoirs have known glucose concentrations, and can be different known glucose concentrations. For example, in some embodiments a first calibration fluid in a first calibration fluid reservoir has a glucose concentration of between about 0 mg/dl and about 100 mg/dl, and a second calibration fluid in a second calibration fluid reservoir has a glucose concentration of between about 100 mg/dl and about 400 mg/dl. The ranges of glucose concentrations in the different calibration fluid reservoirs may, however, be different. When more than one calibration fluid reservoir is used, the calibration fluids in each reservoir may have, however, substantially the same or similar glucose concentrations.

As shown in FIG. 1, the glucose monitor has more than one fluid reservoir. In some embodiments, one of the reservoirs can be filled with a sensing or washing fluid which does not comprise glucose and which is not used to calibrate the glucose sensor. The sensing or washing fluid can comprise, for example, de-ionized water, buffer, surfactants and preservative. In embodiments in which there are two reservoirs and one comprises sensing fluid and the other comprises calibration fluid, the calibration fluid may have a glucose concentration between about 0 mg/dl and about 400 mg/dl, and is used to generate a one-point calibration curve for the sensor. In some embodiments, however, the glucose monitor comprises two or more calibration fluids reservoirs in addition to a sensing or washing fluid reservoir.

One aspect of the invention is monitoring a subject's interstitial fluid glucose concentration. The method can include calibrating the glucose sensor with a plurality of different calibrating fluids with different known glucose concentrations. A first calibration fluid of known glucose concentration is first moved into the sensing area. This can be done, for example, during manufacture of the monitor, prior to the first use by the patient, or any subsequent time when it may be desirable to recalibrate the sensor. The glucose sensor senses glucose in the first calibration fluid in the sensing area and generates an output signal associated with the first known glucose concentration. Any actuating technique described herein may then be used to move a second calibrating fluid with a second known glucose concentration from a second calibration fluid reservoir into the sensing area, displacing the first calibration fluid into the waste area. The sensor then senses the glucose from the second calibration fluid in the sensing area and generates an output signal associated with the second known glucose concentration. Using these at least two associations of known glucose concentration to glucose sensor output, a calibration curve or plot can be used to associate glucose concentration to the output of the glucose sensor, which can then be used to determine glucose concentration of the glucose that diffuses into the sensing area from the patient's interstitial fluid. Any number of calibration fluids, and thus calibration points, can be used to calibrate the glucose sensor. The calibrated sensor is then ready to sense glucose in the sensing area which has diffused from the patient's interstitial fluid.

Describing the method in relation to FIG. 2, upon manual or automatic actuation of actuator 32, fresh calibration fluid is forced from calibration fluid reservoir 12 (only one reservoir is shown) through check valve 34, such as a flap valve, into sensing area 14. Any fluid within the sensing area is generally displaced through second check valve 36 into waste reservoir 16. Check valves or similar gating systems can also be used to prevent contamination.

It may be advantageous to retain a calibration fluid with the lower glucose concentration (such as a first concentration between about 0 mg/dl and 100 mg/dl) in the sensing area after the calibrating step, to provide for faster response times for the glucose sensing. In the method described above where a second calibration fluid has a higher glucose concentration, it may be advantageous to move a volume of the fresh first lower concentration calibration fluid into the sensing area after the glucose sensor has been calibrated. This would move the second sensing fluid from the sensing area into waste reservoir. Alternatively, calibrating can comprise calibrating the sensor with a calibration fluid with a higher glucose concentration followed by calibrating the sensor with a calibration fluid with a lower glucose concentration.

Glucose monitors with more than one calibration reservoir have been described. In such embodiments, the monitor can also include at least one reservoir adapted to house a sensing or washing fluid which does not have any glucose, such as, for example, a buffer, preservative, or de-ionized water. As used herein, “sensing fluid” and “washing fluid” may be used interchangeably. Sensing fluid can be used to displace calibration fluid from the sensing area after the calibration step. Glucose would then diffuse from the patient's interstitial fluid into the sensing fluid which does not contain glucose. This method allows for a glucose concentration determination that does not require factoring the change in glucose concentration from the glucose concentration of a calibration fluid in the sensing area to the glucose concentration in the fluid in the sensing area after diffusion has occurred. This method may therefore provide a simpler, quicker, and more accurate final glucose concentration calculation.

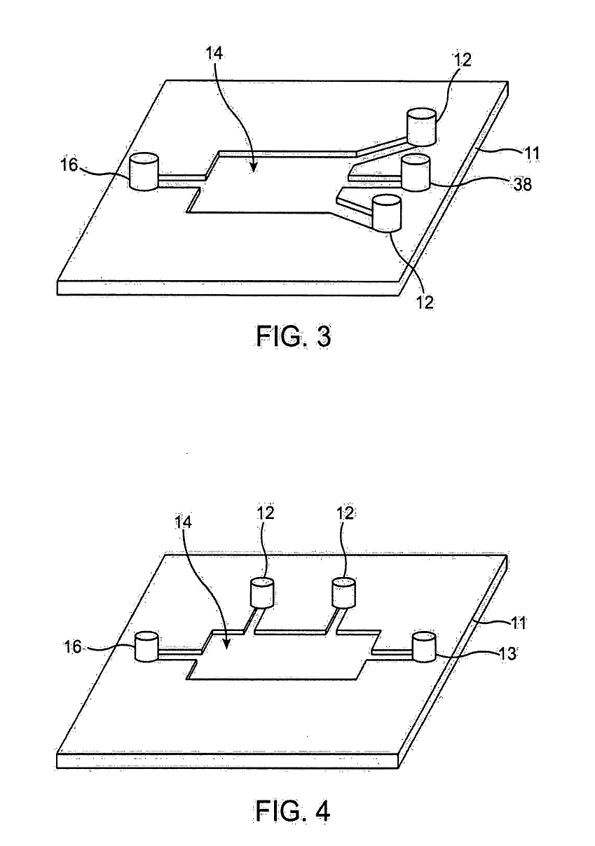

Embodiments in which there are a plurality of calibration fluid reservoirs as well as at least one sensing fluid reservoir are shown in FIGS. 3 and 4. In FIG. 3, glucose monitor 10 is shown comprising two calibration fluid reservoirs 12 and one sensing fluid reservoir 38. All three reservoirs are in fluid communication with the sensing area. An actuator or actuators (not shown in FIG. 3 and 4) can be configured to move fresh fluid from the reservoirs into the sensing area.

In some embodiments the sensor is calibrated with any number of calibration fluids as described herein. The actuator can then move sensing fluid from a sensing fluid reservoir into the sensing area, displacing a calibration fluid. In other embodiments, the sensor may be calibrated with one calibration fluid and then sensing fluid may be moved into the sensing area, followed by a second calibration fluid being moved into the sensing area, displacing the sensing fluid and calibrating the sensor with the second calibrating fluid. Fresh sensing fluid can then be actuated into the sensing area, readying the monitor for diffusion and glucose detection. In this method, there is a “wash” step between calibrating the sensor with fluids of different known glucose concentrations.

In some embodiments at least one finger-stick calibration may optionally be performed or may be required to be performed at any point during the use of the monitors described herein.

Waste reservoirs may be or include an absorption device such as a wicking material to absorb waste fluids. In such embodiments the waste reservoir may not necessarily be an enclosed structure, but may simply be a wicking material or substance in fluid communication with the sensing area so that it can wick waste fluids as they are moved from the sensing area.

While in some embodiments the glucose monitor may be manually actuated to initiate the calibrating procedure, the glucose monitor can also be self-calibrating or self-actuating. For example, the glucose monitor can include a programmable component, such as a timer, that is programmed to automatically activate an actuator, such as a pump and valve system, to initiate the flow of fresh fluid from any of the fluid reservoirs into the sensing area. The timer can be preprogrammed, or in some embodiments the monitor also includes a remote device that is separate from the sensor that can display a glucose concentration. The remote device can be adapted such that it can program the programmable component. For example, a patient may want to program the monitor to calibrate itself at certain times during the day. The monitor can include a timer that can be programmed, reprogrammed by the patient, and/or automatically reprogrammed. The remote device can be adapted for manual programming.

In some embodiments the glucose monitor includes a body and sensing area temperature sensor, which is more fully described in co-assigned U.S. patent application Ser. No. 11/642,196, filed Dec. 20, 2006.

In some embodiments the glucose monitor includes a vibration assembly adapted to ease the penetration of the needle into the stratum corteum of the skin. Description of exemplary vibration assemblies are described in co-assigned U.S. patent application Ser. No. 11/642,196, filed Dec. 20, 2006.

In some embodiments the monitor can include an applicator to apply the sensor pad or adhesive pad to the skin. The applicator pad may be part of the sensor device or when the monitor includes separate components, it may be included in any of the different components.

In some embodiments, the tissue piercing elements, fluid reservoirs, sensing area, sensor, and optional adhesive pads are contained within a sensing structure separate from a reusable structure comprising the electronics element and actuator. This configuration permits the sensing structure, comprising the sensor, sensing fluid and tissue piercing elements to be discarded after a period of use (e.g., when the fluid reservoirs are depleted) while enabling the reusable structure comprising the electronics and actuator to be reused. A flexible covering (made, e.g., of polyester or other plastic-like material) may surround and support the disposable structure. In particular, the interface between an actuator and a fluid reservoir permits the actuator to move fluid out of the reservoir, such as by deforming a wall of the reservoir or forcing the fluid out of the reservoir using a pressurized mechanism, such as a piston. In these embodiments, the disposable sensing structure and the reusable structure may have a mechanical connection, such as a snap or interference fit. Any of the monitor components described herein may, however, be located in the reusable structure or the sensing structure. For example, the tissue piercing elements could be configured to be located in the reusable structure. As another example, one or more fluid reservoirs may be located in the reusable structure and may be refillable, emptyable or separately replaceable from other disposable structures.

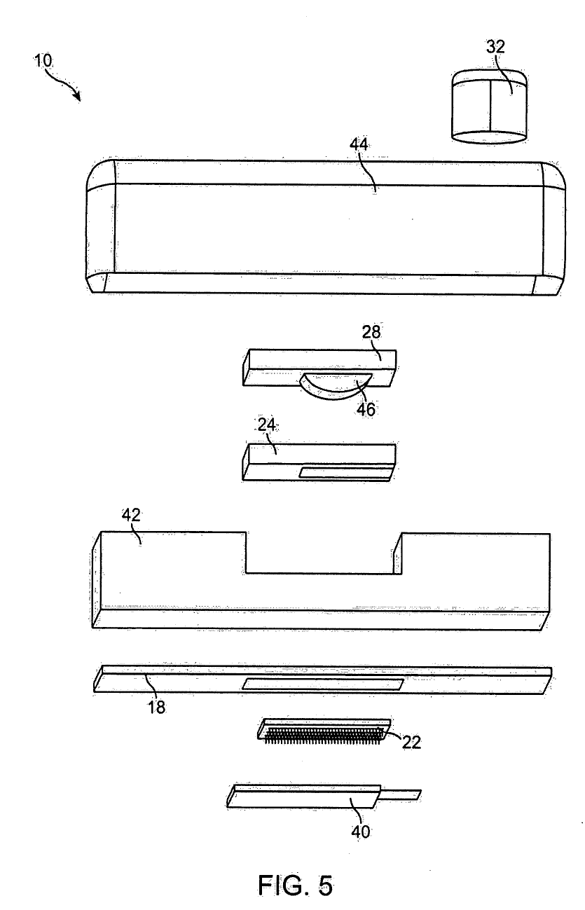

FIG. 5 shows an exploded view of another embodiment of the invention. This figure shows a removable seal 40 covering the distal end of tissue piercing elements 22 and attached, e.g., by adhesive. Removable seal 40 retains the fluid within the tissue piercing elements and sensing area prior to use and is removed prior to placing the glucose monitor 10 on the skin using adhesive seal 18. In this embodiment, tissue piercing elements 22, the fluid and waste reservoirs, sensing area 14 and sensor 24 are contained within and/or supported by sensing structure 42 which can be a disposable portion of the monitor. Reusable structure 44 comprises or supports electronics element 28 and actuator 32 that can be used to move sensing fluid out of the fluid reservoirs, through the sensing area into the waste reservoir. Electrical contacts 46 extend from electronics element 28 to make contact with, for example, electrodes in glucose sensor 24 when the device is assembled.

The following is a description of glucose sensors that may be used with the glucose monitors of this invention. In 1962 Clark and Lyons proposed the first enzyme electrode (that was implemented later by Updike and Hicks) to determine glucose concentration in a sample by combining the specificity of a biological system with the simplicity and sensitivity of an electrochemical transducer. The most common strategies for glucose detection are based on using either glucose oxidase or glucose dehydrogenase enzyme.

Electrochemical sensors for glucose, based on the specific glucose oxidizing enzyme glucose oxidase, have generated considerable interest. Several commercial devices based on this principle have been developed and are widely used currently for monitoring of glucose, e.g., self testing by patients at home, as well as testing in physician offices and hospitals. The earliest amperometric glucose biosensors were based on glucose oxidase (GOX) which generates hydrogen peroxide in the presence of oxygen and glucose according to the following reaction scheme:

Glucose+GOX-FAD (ox)→Gluconolactone+GOX-FADH₂ (red)

GOX-FADH₂ (red)+O₂→GOX-FAD (ox)+H₂O₂

Electrochemical biosensors are used for glucose detection because of their high sensitivity, selectivity and low cost. In principal, amperometric detection is based on measuring either the oxidation or reduction of an electroactive compound at a working electrode. A constant potential is applied to that working electrode with respect to another electrode used as the reference electrode. The glucose oxidase enzyme is first reduced in the process but is reoxidized again to its active form by the presence of any oxygen resulting in the formation of hydrogen peroxide. Glucose sensors generally have been designed by monitoring either the hydrogen peroxide formation or the oxygen consumption. The hydrogen peroxide produced is easily detected at a potential of 0.0 volts, 0.1 volts, 0.2 volts, or any other fixed potential relative to a reference electrode such as a Ag/AgCl electrode. However, sensors based on hydrogen peroxide detection are subject to electrochemical interference by the presence of other oxidizable species in clinical samples such as blood or serum. On the other hand, biosensors that monitor oxygen consumption are affected by the variation of oxygen concentration in ambient air or in any of the fluids used with the monitors as described herein. In order to overcome these drawbacks, different strategies have been developed and adopted.

Selectively permeable membranes or polymer films have been used to suppress or minimize interference from endogenous electroactive species in biological samples. Another strategy to solve these problems is to replace oxygen with electrochemical mediators to reoxidize the enzyme. Mediators are electrochemically active compounds that can reoxidize the enzyme (glucose oxidase) and then be reoxidized at the working electrode as shown below:

GOX-FADH₂ (red)+Mediator (ox)→GOX-FAD (ox)+Mediator (red)

Organic conducting salts, ferrocene and ferrocene derivatives, ferricyanide, quinones, and viologens are considered good examples of such mediators. Such electrochemical mediators act as redox couples to shuttle electrons between the enzyme and electrode surface. Because mediators can be detected at lower oxidation potentials than that used for the detection of hydrogen peroxide the interference from electroactive species (e.g., ascorbic and uric acids present) in clinical samples such as blood or serum is greatly reduced. For example ferrocene derivatives have oxidation potentials in the +0.1 to 0.4 V range. Conductive organic salts such as tetrathiafulvalene-tetracyanoquinodimethane (TTF-TCNQ) can operate as low as 0.0 Volts relative to a Ag/AgCl reference electrode. Nankai et al, WO 86/07632, published Dec. 31, 1986, discloses an amperometric biosensor system in which a fluid containing glucose is contacted with glucose oxidase and potassium ferricyanide. The glucose is oxidized and the ferricyanide is reduced to ferrocyanide. This reaction is catalyzed by glucose oxidase. After two minutes, an electrical potential is applied, and a current caused by the re-oxidation of the ferrocyanide to ferricyanide is obtained. The current value, obtained a few seconds after the potential is applied, correlates to the concentration of glucose in the fluid.

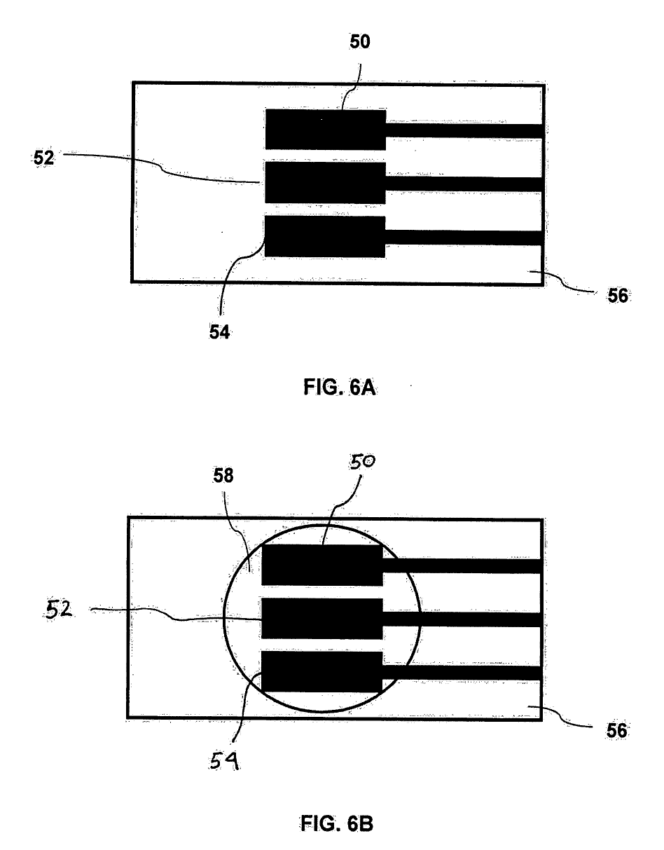

There are multiple glucose sensors that may be used with this invention. In a three electrode system, shown in FIGS. 6A and 6B a working electrode 50, such as Pt, C, or Pt/C is referenced against a reference electrode 52 (such as Ag/AgCl) and a counter electrode 54, such as Pt, provides a means for current flow. The three electrodes are mounted on an electrode substrate 56 as shown in FIG. 6A, then covered with a reagent 58 as shown in FIG. 6B.

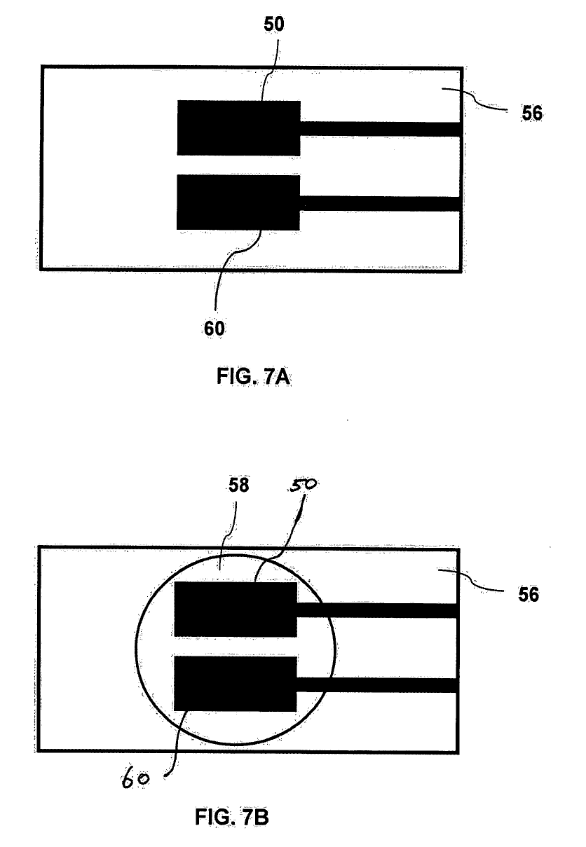

FIGS. 7A and 7B show a two electrode system, wherein the working and auxiliary electrodes, 50 and 60 respectively, are made of different electrically conducting materials. Like the embodiment of FIGS. 6A and 6B, the electrodes are mounted on a flexible substrate 56 (FIG. 7A) and covered with a reagent 58 (FIG. 7B). In an alternative two electrode system, the working and auxiliary electrodes are made of the same electrically conducting materials, where the reagent exposed surface area of the auxiliary electrode is slightly larger than that of the working electrode or where both the working and auxiliary electrodes are substantially of equal dimensions.

In amperometric and coulometric biosensors, immobilization of the enzymes is also very important. Conventional methods of enzyme immobilization include covalent binding, physical adsorption or cross-linking to a suitable matrix may be used. In some embodiments the reagent chemistry can be deposited away from the electrodes using various different dispensing methods.

The glucose sensor can be constructed by immobilizing glucose oxidase enzyme on top of the electrode by using a proprietary cross linker and a coating membrane. The cross linker will hold the enzyme on top of the sensor, and the thin layer membrane (e.g., Nafion, cellulose acetate, polyvinyl chloride,urethane etc) will help the long term stability of the glucose sensor. In the presence of oxygen the glucose oxidase will produce hydrogen peroxide. The hydrogen peroxide can be readily oxidized at the working electrode surface in either two or three electrodes systems

In some embodiments, the reagent is contained in a reagent well in the biosensor. The reagent includes a redox mediator, an enzyme, and a buffer, and covers substantially equal surface areas of portions of the working and auxiliary electrodes. When a sample containing the analyte to be measured, in this example glucose, comes into contact with the glucose biosensor the analyte is oxidized, and simultaneously the mediator is reduced. After the reaction is complete, an electrical potential difference is applied between the electrodes. In general the amount of oxidized form of the redox mediator at the auxiliary electrode and the applied potential difference must be sufficient to cause diffusion limited electrooxidation of the reduced form of the redox mediator at the surface of the working electrode. After a short time delay, the current produced by the electrooxidation of the reduced form of the redox mediator is measured and correlated to the amount of the analyte concentration in the sample. In some cases, the analyte sought to be measured may be reduced and the redox mediator may be oxidized.

In the present invention, these elements are satisfied by employing a readily reversible redox mediator and using a reagent with the oxidized form of the redox mediator in an amount sufficient to insure that the diffusion current produced is limited by the oxidation of the reduced form of the redox mediator at the working electrode surface. For current produced during electrooxidation to be limited by the oxidation of the reduced form of the redox mediator at the working electrode surface, the amount of the oxidized form of the redox mediator at the surface of the auxiliary electrode exceeds the amount of the reduced form of the redox mediator at the surface of the working electrode. Importantly, when the reagent includes an excess of the oxidized form of the redox mediator, as described below, the working and auxiliary electrodes may be substantially the same size or unequal size as well as made of the same or different electrically conducting material or different conducting materials. From a cost perspective the ability to utilize electrodes that are fabricated from substantially the same material represents an important advantage for inexpensive biosensors.

As explained above, the redox mediator must be readily reversible, and the oxidized form of the redox mediator must be of sufficient type to receive at least one electron from the reaction involving enzyme, analyte, and oxidized form of the redox mediator. For example, when glucose is the analyte to be measured and glucose oxidase is the enzyme, ferricyanide or quinone may be the oxidized form of the redox mediator. Other examples of enzymes and redox mediators (oxidized form) that may be used in measuring particular analytes by the present invention are ferrocene and or ferrocene derivative, ferricyanide, and viologens. Buffers may be used to provide a preferred pH range from about 4 to 8. In one embodiment, the pH range is from about 6 to 7. The buffer may be phosphate (e.g., potassium phosphate) and may be in a range from about 0.01M to 0.5M, such as about 0.05M. (These concentration ranges refer to the reagent composition before it is dried onto the electrode surfaces.) More details regarding glucose sensor chemistry and operation may be found in: Clark L C and Lyons C, “Electrode Systems for Continuous Monitoring in Cardiovascular Surgery,” Ann NY Acad Sci, 102:29, 1962; Updike S J, and Hicks G P, “The Enzyme Electrode,” Nature, 214:986, 1967; Cass, A. E. G., G. Davis. G. D. Francis, et. al. 1984. Ferrocene—mediated enzyme electrode for amperometric determination of glucose. Anal. Chem. 56:667-671; and Boutelle. M. G., C. Stanford. M. Fillenz. et al. 1986. An amperometric enzyme electrode for monitoring brain glucose in the freely moving rat. Neurosci lett. 72:283-288.

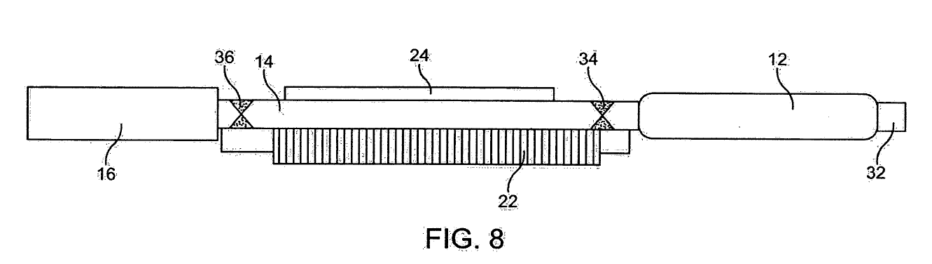

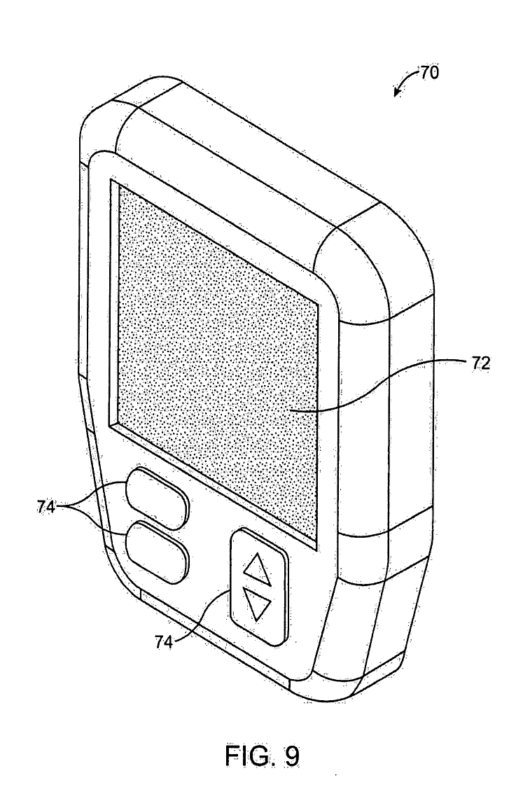

An alternative embodiment of the disposable portion of the glucose monitor invention is shown in the side sectional view in FIG. 8 with tissue piercing elements 22 and a glucose sensor 24 in fluid communication with a sensing area 14. In this embodiment, actuator 32 is on calibration fluid reservoir 12, and the waste reservoir 16 can be expandable. Operation of actuator 32 moves calibration fluid (or sensing fluid) from reservoir 12 through one way check valve 34 into the sensing area 14 and forces fluid within sensing area through check valve 36 into the optionally expandable waste reservoir 16.

In some of the embodiments described herein, the starting amount or fresh fluid in a calibration fluid reservoir or a sensing fluid reservoir is about 2.5 ml or less, and operation of an actuator moves about 5 μL to about 50 μL of fresh fluid into the sensing channel.

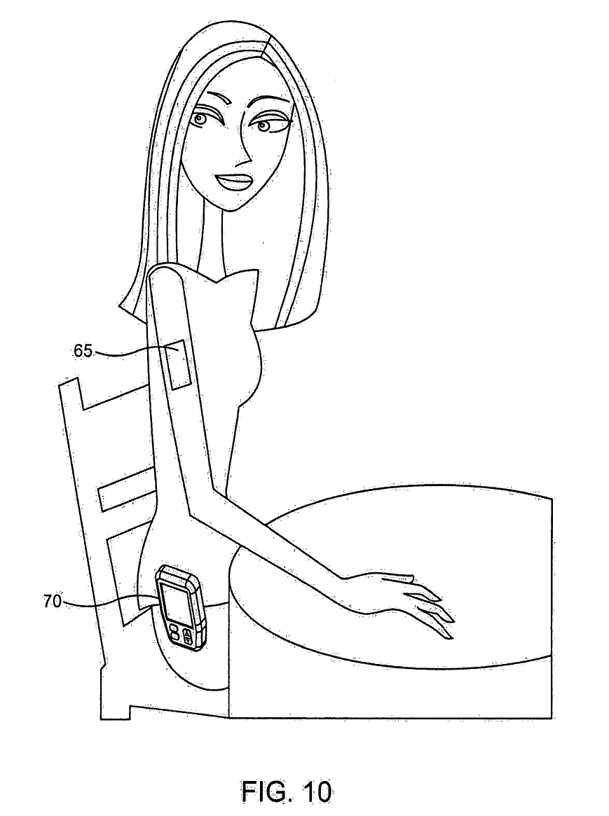

FIGS. 9 and 10 show a glucose monitor comprising a sensing device 65 and remote device 70. The remote device can be configured to be worn by a patient on a belt, or carried in a pocket or purse. In this embodiment, glucose sensor information is transmitted from the sensing device 65 to remote device 70 using, e.g., wireless communication such as radio frequency (RF) or Bluetooth wireless technology. The remote device may maintain a continuous link with the sensor, or it may periodically receive information from the sensor. The sensing device and the remote device may be synchronized using RFID technology or other unique identifiers.

Remote device may be provided with a display 72 and user controls 74. The display may show, e.g., glucose values, directional glucose trend arrows and rates of change of glucose concentration. The remote device can also be configured with a speaker or vibrator adapted to deliver an audible alarm, such as high and low glucose alarms. Additionally, the remote device can include a memory configured to store glucose data for analysis by the user or by a health care provider.

At least one power source, such as a battery, will be required to supply power to the monitor. The glucose monitor may comprise one power source for the entire monitor, or may comprise more than one power source that each provide power to any number of different components in the glucose monitor. For example, one power source may supply power to a sensor and a transmitter, or separate power sources may supply power to a sensor and a transmitter. An important advantage of the transmitter is that the transmitter is fabricated without a battery as a power source or it can be made containing a rechargeable battery.

In FIG. 9 and 10, the sensing device and the remote device can comprise their own power sources and may comprise any number of power sources. The sensing device may comprise a disposable portion and a reusable portion as described herein. The disposable portion can include a power source that supplies power to components in the disposable portion only, or to components in the reusable portion as well. For example, a power source in the disposable portion of the sensing device can supply power to a sensor in the disposable portion and to a transmitter in the reusable portion of the sensing device. Either when the disposable portion is to be discarded or the power source runs out of power, the disposable portion can be replaced with a new disposable portion, which will include a new power source. Thus, the life of a transmitter in the reusable portion will not be limited by the life of a power source such as a battery which can be easily replaced without requiring a new transmitter to be used. Rechargeable power sources may also be used.

The monitor, and preferably the remote device, can be programmed with high and low threshold levels such that when the patient's glucose levels are higher than the high threshold level or lower than the low threshold level the monitor will alert the patient or a third party. The remote device can be preprogrammed to default threshold levels, can be manually programmed using, for example, the remote device's user interface, or the remote device can be adapted to dynamically adjust threshold levels based on, for example, current glucose concentrations, trends in the glucose concentrations, or user inputs into the remote device such as an indication from the user that she is going to sleep or about to consume food. The alert can occur based on any method to alert the patient, such as, for example, with an audible alert like a beep, a visual alert such as a blinking light, or mechanical alert such as vibrating. The monitor can also be adapted to wirelessly alert a device separate from the remote device, such as a health care provider or parent when the glucose concentration is above or below the threshold levels, or trending below or above the threshold levels. The monitor, and preferably the remote device, can also be adapted to display glucose concentration trends and can alert the patient when the concentration is trending down or up. Trends can be stored in the remote device and can be used to dynamically adjust the threshold levels. The device can also include external data download capability

In some embodiments, the source reservoir for the calibration and sensing fluid may be in a blister pack which maintains its integrity until punctured or broken. The actuator may be a small syringe or pump. Use of the actuator for recalibration of the sensor may be performed manually by the user or may be performed automatically by the device if programmed accordingly. There may also be a spring or other loading mechanism within the reusable housing that can be activated to push the disposable portion—and specifically the tissue piercing elements Downward into the user's skin.

While exemplary embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An analyte monitor, comprising:
a plurality of tissue piercing elements each having a distal opening, a proximal opening, and an interior lumen extending between the distal and proximal openings;
a sensing area in fluid communication with the proximal openings of the plurality of tissue piercing elements;
a plurality of calibration fluid reservoirs each adapted to house a calibration fluid, wherein the plurality of calibration fluid reservoirs are in fluid communication with the sensing area; and
a sensor configured to detect an analyte and provide an output indicative of the analyte concentration of a fluid in the sensing area.

2. The monitor of claim 1 wherein the plurality of calibration fluid reservoirs comprise a first calibration fluid reservoir adapted to house a first calibration fluid and a second calibration fluid reservoir adapted to house a second calibration fluid.

3. The monitor of claim 2 wherein the first calibration fluid and the second calibration fluid have different known concentrations of the analyte.

4. The monitor of claim 3 wherein the first calibration fluid has a glucose concentration between about 0 mg/dl and about 100 mg/dl and the second calibration fluid has a glucose concentration of between about 100 mg/dl and about 400 mg/dl.

5. The monitor of claim 1 further comprising an actuator configured to move the calibration fluids from the plurality of calibration fluid reservoirs into the sensing area.

6. The monitor of claim 5 wherein the actuator comprises at least one pump.

7. The monitor of claim 6 further comprising a plurality of valves configured to facilitate the movement of the calibration fluids unidirectionally from the plurality of calibration fluid reservoirs into the sensing area.

8. The monitor of claim 5 wherein the actuator is configured to be manually actuated.

9. The monitor of claim 5 wherein the actuator is configured to be automatically actuated.

10. The monitor of claim 9 wherein the monitor further comprises a programmable component in communication with the actuator wherein the programmable component is programmed to automatically actuate the actuator.

11. The monitor of claim 10 wherein the monitor further comprises a remote device and wherein the programmable component is disposed in a housing with the sensor, wherein the programmable component in configured to be wirelessly programmed using the remote device.

12. The monitor of claim 10 wherein the monitor further comprises a remote device and wherein the programmable component is disposed in the remote device, the programmable component is configured to be programmed using the remote device, and wherein the programmable component is configured to be in wireless communication with the actuator to automatically actuate the actuator.

13. The monitor of claim 5 wherein the actuator is configured to move a first calibration fluid with a first known analyte concentration from a first calibration fluid reservoir into the sensing area and then move a second calibration fluid with a second known analyte concentration from a second calibration fluid reservoir into the sensing area, thereby displacing the first calibration fluid from the sensing area.

14. The monitor of claim 13 wherein the sensor is configured to detect the analyte in the first and second calibration fluids when in the sensing area, the monitor further comprising a memory to store sensor calibration data, the sensor calibration data comprising the first and second known analyte concentrations and a first output and a second output from the sensor indicative of the first and second known analyte concentrations.

15. The monitor of claim 14 wherein the monitor further comprises a remote device, the memory disposed in the remote device.

16. The monitor of claim 15 further comprising a transmitter configured to transmit an output from the sensor indicative of the analyte that has diffused from the patient's interstitial fluid into the sensing area to a receiver disposed in the remote device, the remote device further comprising a processor adapted to determine an analyte concentration based on the output from the sensor and the sensor calibration stored in the memory.

17. The monitor of claim 16, wherein the transmitter is either fabricated without a power source or the transmitter comprises a rechargeable power source.

18. The monitor of claim 16 further comprising a display adapted to display the analyte concentration determined by the processor.

19. The monitor of claim 18 wherein the display is disposed in the remote device.

20. The monitor of claim 18 wherein the analyte concentration is the patient's blood glucose concentration.

21. The monitor of claim 1 further comprising at least one waste reservoir in fluid communication with the sensing area adapted to receive fluid moved from the sensing area.

22. The monitor of claim 1 further comprising a housing comprising a disposable portion and reusable portion, the disposable portion being adapted to support the plurality of tissue piercing elements, the plurality of calibration fluid reservoirs, the sensing area, and at least part of the analyte sensor, the reusable portion comprising an electrical connection to the at least part of the analyte sensor in the disposable portion, the housing further comprising a connector adapted to connect and disconnect the disposable portion from the reusable portion.

23. The monitor of claim 1 further comprising a sensing fluid reservoir in fluid communication with the sensing area, wherein the sensing fluid reservoir is adapted to house a sensing fluid which does not comprise the analyte.

24. The monitor of claim 23 wherein the sensing fluid comprises at least one of the group consisting of de-ionized water, buffer, and preservative.

25. The monitor of claim 23 further comprising an actuator configured to move fluid from the plurality of calibration reservoirs and the sensing reservoir into the sensing area.

26. The monitor of claim 25 wherein the actuator comprises at least one pump.

27. The monitor of claim 25 wherein the actuator is configured to be manually actuated.

28. The monitor of claim 25 wherein the actuator is configured to be automatically actuated.

29. The monitor of claim 28 wherein the actuator is further configured to automatically first move a first calibration fluid from a first calibration reservoir into the sensing area and then automatically move a second calibration fluid from a second calibration reservoir into the sensing area, thereby displacing the first calibration fluid from the sensing area, and then automatically move sensing fluid from the sensing fluid reservoir into the sensing area, thereby displacing the second calibration fluid from the sensing area.

30. The monitor of claim 29 wherein the sensor is configured to detect the analyte in the first and second calibration fluids when in the sensing area, the monitor further comprising a memory to store a sensor calibration, the sensor calibration comprising the first and second known analyte concentrations and a first output and a second output from the sensor indicative of the first and second known analyte concentrations.

31. The monitor of claim 1 further comprising
a transmitter adapted to transmit the output indicative of the analyte concentration of the fluid in the sensing area to a remote device; at least one power source, a reusable portion comprising the transmitter; a disposable portion comprising the at least one power source, wherein the at least one power source is adapted to be disposable and wherein the transmitter is adapted to be reusable.

32. The monitor of claim 1 wherein the analyte is glucose.

33. A method of monitoring a concentration of an analyte in a patient's interstitial fluid in vivo, the method comprising:
calibrating an analyte monitor, the analyte monitor comprising:
a plurality of tissue piercing elements each having a distal opening, a proximal opening, and an interior lumen extending between the distal and proximal openings;
a sensing area in fluid communication with the proximal openings of the plurality of tissue piercing elements;
a plurality of calibration fluid reservoirs each adapted to house a calibration fluid, wherein the plurality of calibration fluid reservoirs are in fluid communication with the sensing area;
a sensor configured to detect the analyte and provide an output indicative of the analyte concentration of a fluid in the sensing area;
at least one waste reservoir in fluid communication with the sensing area configured to receive fluid from the sensing area; and
a memory in communication with the sensor;
wherein calibrating the analyte monitor comprises:
moving a first calibration fluid with a first known analyte concentration from a first calibration reservoir into the sensing area;
sensing an analyte concentration in the first calibration fluid while in the sensing area with the analyte sensor, the sensor providing a first output indicative of the analyte concentration of the first calibrating fluid;
moving a second calibration fluid with a second known analyte concentration from a second calibration reservoir into the sensing area thereby displacing the first calibration fluid with the second calibration fluid into the at least one waste reservoir;
sensing an analyte concentration in the second calibration fluid while in the sensing area with the analyte sensor, the sensor providing a second output indicative of the analyte concentration of the second calibrating fluid; and
storing a sensor calibration in the memory, the sensor calibration comprising an association between the first and second known analyte concentrations and the first and second outputs indicative of the first and second known analyte concentrations.

34. The method of claim 33 wherein the second calibration fluid is a sensing fluid that does not comprise the analyte, and wherein moving the sensing fluid into the sensing area comprises washing the sensing area with the sensing fluid.

35. The method of claim 34 wherein the sensing fluid comprises at least one of the group consisting of de-ionized water, buffer, and preservative.

36. The method of claim 33 further comprising piercing only as deep as into the epidermis layer of a patient or user's skin with the plurality of tissue piercing elements.

37. The method of claim 36 wherein piercing only as deep as into the epidermis layer of the patient or user's skin with the plurality of tissue piercing elements allows diffusion of the analyte from the patient's interstitial fluid through the plurality of tissue piercing elements and into the sensing area substantially without extracting interstitial fluid through the plurality of tissue piercing elements.

38. The method of claim 37 further comprising sensing the analyte concentration of the diffused analyte using the sensor and determining the patient's analyte concentration using the sensor calibration stored in the memory.

39. The method of claim 38 wherein the monitor further comprises a remote device, and wherein the memory is disposed in a remote device, the method further comprising wirelessly transmitting the outputs from the sensor to the remote device before determining the patient's analyte concentration.

40. The method of claim 38 wherein the method further comprises displaying the determined analyte concentration.

41. The method of claim 40 wherein displaying the determined analyte concentration comprises displaying the determined analyte concentration using a remote device.

42. The method of claim 38 further comprising moving a sensing fluid which does not comprise the analyte from a sensing fluid reservoir into the sensing area thereby displacing the second calibration fluid with the sensing fluid into the at least one waste reservoir, wherein moving the sensing fluid occurs before the piercing step.

43. The method of claim 38 further comprising recalibrating the sensor after determining the patient or user's analyte determination.

44. The method of claim 33 wherein moving the first and second calibration fluids comprises actuating an actuator.

45. The method of claim 44 wherein actuating the actuator comprising automatically actuating the actuator.

46. The method of claim 45 further comprising programming the monitor to automatically actuate the actuator.

47. The method of claim 46 wherein the monitor comprises a remote device and programming the monitor to automatically actuate the actuator comprises programming the monitor using the remote device.

48. The method of claim 44 further comprising recalibrating the sensor, wherein recalibrating comprises actuating the actuator.

49. The method of claim 44 wherein actuating the actuator comprises manually actuating the actuator.

50. The method of claim 33 wherein the analyte is glucose.
